(19) United States
(12) Patent Application Publication
(10) Pub. No.: US 2017/0285618 A1
(43) Pub. Date: Oct. 5, 2017

(54) SYSTEMS AND METHODS FOR INTEGRATED ROBUST ORTHOTICS AND PROSTHETICS HYPER-CUSTOMIZATION

(71) Applicant: UNIVERSITY OF CENTRAL FLORIDA RESEARCH FOUNDATION, INC., Orlando, FL (US)

(72) Inventors: Bradley Rosenkrantz, Orlando, FL (US); Sam Drucker, Orlando, FL (US); Sheikh Salauddin, Orlando, FL (US); Brandon Lowe, Orlando, FL (US)

(21) Appl. No.: 15/478,489

(22) Filed: Apr. 4, 2017

Related U.S. Application Data
(60) Provisional application No. 62/317,790, filed on Apr. 4, 2016.

(51) Int. Cl. G05B 19/4099; A61F 2/50

(52) U.S. Cl. CPC G05B 19/4099; A61F 2/5046; A61F 2002/505; G05B 2219/49023

(57) ABSTRACT

Disclosed are systems, methods and computer-readable medium for developing a custom design for a prosthetic for a residual limb comprising: measuring, using a scanning apparatus, a usable measurement of the residual limb; and creating, using a computing device, a design for a prosthetic for at least a portion of a missing portion of the residual limb, wherein the prosthetic is proportionally sized according to a correlation between the usable measurement and normal anthropometric measurements.

### CROSS REFERENCE TO RELATED APPLICATION

This application claims priority to and benefit of U.S. Provisional Patent Application No. 62/317,790 filed Apr. 4, 2016, which is fully incorporated by reference and made a part hereof.

### BACKGROUND

Since 2005, over 1.6 million Americans have been living with limb loss. Globally, over 29 million people are in need of prosthetics to treat limb loss. Approximately one third of these cases involve the loss of an upper extremity. Due to the complex functionality of the upper limb, this loss tends to be more life altering. Upper limb prosthetics have shown to increase the quality of life of the patient as well as increase functionality. These prosthetics can be simply cosmetic or functional, and can harness the electrical impulses still present in the residual limb. Recently, three-dimensional (3D) printing has dramatically decreased the cost of designing and producing these prosthetics. Currently, CAD scanners are available that can determine anatomical constraints. However, these scanners can cost up to $10,000 and are not user friendly. Current CAD scanners focus on producing more comfortable, higher end prosthetics as opposed to streamlining the production of low cost prosthetics. Further, CAD scanners are designed to determine the total volume of a residual limb, which may not be necessary for designing a prosthetic.

Therefore, systems and methods are desired that overcome challenges in the art, some of which are described above.

### SUMMARY

Disclosed herein are embodiments of a scanner system and method that can be used to determine a usable measurement of a residual limb. A correlation between an interval measured usable measurement and the normal size of the missing portion of the residual limb is also disclosed and used for the design of a prosthetic, including 3D printed prosthetics. The disclosed systems and methods allow increased production and delivery of low cost prosthetics.

Disclosed herein are methods for developing a custom design for a prosthetic for a residual limb. One method comprises measuring, using a scanning apparatus, a usable measurement of the residual limb; and creating, using a computing device, a design for a prosthetic for at least a portion of a missing portion of the residual limb, wherein the prosthetic is proportionally sized according to a correlation between the usable measurement and normal anthropometric measurements. The scanning apparatus may comprise a rigid arm, wherein one or more measurement sensors are mounted on the rigid arm and said rigid arm with said one or more measurement sensors mounted thereon encircles the residual limb to measure the usable measurement. For examples, the one or more measurement sensors may comprise one or more time of flight (TOF) sensors. The scanning apparatus may be communicatively coupled with the computing device such that the usable measurement is transmitted from the scanning apparatus to the computing device. The usable measurement may be, for example, a diameter of the residual limb. As a non-limiting example, the usable measurement comprises the diameter of a forearm and the prosthetic comprises at least a hand that is proportionally sized according to a correlation between the forearm diameter and hand size.

Alternatively or optionally, the method may comprise the residual limb being placed in a stand before measuring, using the scanning apparatus, the usable measurement of the residual limb.

Alternatively or optionally, the method may comprise the computing device executing computer-readable instructions to create the designed prosthetic using a 3-dimensional (3D) printer.

Alternatively or optionally, the method may comprise the computing device executing computer-readable instructions to adjust the usable measurement of the residual limb by a constant distance offset value to allow for growth of the residual limb.

Also disclosed herein are systems for developing a custom design for a prosthetic for a residual limb. The system may comprise a scanning apparatus, wherein the scanning apparatus determines a usable measurement of the residual limb; and a computing device comprising a processor and a memory, the processor executing computer-readable instructions stored in the memory, said computer-readable instructions causing the processor to: receive the usable measurement from the scanning apparatus; and create a design for a prosthetic for at least a portion of a missing portion of the residual limb, wherein the prosthetic is proportionally sized according to a correlation between the usable measurement and normal anthropometric measurements. The scanning apparatus may comprise a rigid arm, wherein one or more measurement sensors are mounted on the rigid arm and the rigid arm with the one or more measurement sensors mounted thereon encircles the residual limb to measure the usable measurement. In one example, the one or more measurement sensors comprise one or more time of flight (TOF) sensors. The usable measurement may comprise a diameter of the residual limb. For example, the usable measurement may comprise the diameter of a forearm and the processor executes computer-readable instructions to design a prosthetic that comprises at least a hand that is proportionally sized according to a correlation between the forearm diameter and hand size.

Alternatively or optionally, the system may further comprise a stand, wherein the residual limb is placed in a stand before measuring, using the scanning apparatus, the usable measurement of the residual limb.

Also alternatively or optionally, the system may further comprise a three-dimensional (3D) printer, wherein the processor executes computer-readable instructions to create the designed prosthetic using the 3D printer.

Alternatively or optionally, the system may further comprise the processor executing computer-readable instructions to adjust the usable measurement of the residual limb by a constant distance offset value to allow for growth of the residual limb.

Further disclosed herein is a non-transitory computer-readable medium having computer executable instructions stored thereon. The computer executable instructions cause a processor to perform a method of developing a custom design for a prosthetic for a residual limb. One embodiment of the method comprises receiving a usable measurement of a residual limb from a scanning apparatus; and creating a design for a prosthetic for at least a portion of a missing portion of the residual limb, wherein the prosthetic is proportionally sized according to a correlation between the usable measurement and normal anthropometric measurements.

Alternatively or optionally, the non-transitory computer-readable medium may further comprise computer-readable instructions to create the designed prosthetic using a 3-dimensional (3D) printer.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems:

FIGS. 1A and 1B illustrate an exemplary overview system for scanning a residual limb and socket/prosthetic for hyper-customization methods;

FIG. 2 illustrates an example of a stand that can be used with the system shown in FIGS. 1A and 1B, though other stand designs are contemplated within the scope of this disclosure;

FIG. 3 illustrates an exemplary scanner apparatus comprising one or more distance sensors mounted at fixed increments along a rigid arm which is oriented parallel in relation to the residual limb;

FIG. 4 is a graphical illustration of a correlation made between the diameter of the forearm and the normal size of a human hand as a function of age;

FIG. 5 is a flowchart that illustrates an exemplary method for developing a custom design for a prosthetic for a residual limb; and

FIG. 6 illustrates an exemplary computer that can be used for developing a custom design for a prosthetic for a residual limb.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

The present methods and systems may be understood more readily by reference to the following detailed description of preferred embodiments and the Examples included therein and to the Figures and their previous and following description.

Since 2005, over 1.6 million Americans have been living with limb loss, one third of which is an upper extremity loss. Peripheral vascular disease accounts for the cause of a majority of these cases, however traumatic injury is more prevalent in those under 45 years of age. Additionally, 15 out of 100,000 newborns are affected with a congenital upper limb abnormality. Due in part to an aging population, current estimates suggest that this number will more than double by 2050 without intervention. This may be attributed to the increasing prevalence of vascular diseases such as complications resulting from diabetes. Globally, this number is significantly higher. In low and middle income countries, over 29 million people are in of need prosthetics or orthotics to treat limb loss. Compared to a lower limb amputation, upper limb amputations tend to be more life altering. This is likely due to the complex anatomy and functionality of the upper limb.

Loss of the upper limb can be classified into six major classes. Wrist disarticulation involves the loss of the hand at the radiocarpal joint. Forearm amputation, also referred to as transradial, refers to an amputation which involves the hand as well as part of the forearm. Elbow disarticulation involves the complete loss of the forearm while transhumeral amputation also includes a partial amputation of the humerus. Shoulder disarticulation involves loss of the arm at the glenohumeral joint where forequarter amputation involves resection of the arm including part of the scapula and clavicle. Each of these amputations confer different criteria for designing prosthetics, including the use of support straps, the development of comfortable sockets, and the terminal end device.

Utilization of an upper limb prosthetic has been shown to both increase the quality of life for the patient as well as increase the functionality of the residual limb. A variety of different prosthetics can be utilized to treat a patient with an upper limb amputation or abnormality. The prostheses can be functional, cosmetic, or both. Functional prosthetics can be subdivided into cable controlled or externally powered. A cable controlled prosthetic relies on body movements independent of the residual limb to allow for functionality of the prosthetic.

Externally powered prosthetics can be myoelectric, switch, or servo controlled. A myoelectric prosthetic relies on the electromagnetic signal that can be measured upon contraction of the residual muscles of the affected limb. For example, a myoelectric prosthetic can be used to treat a wrist disarticulation where the residual flexors of the anterior forearm can function in controlling flexion of the fingers. Advanced myoelectric prosthetics even allow for the control of individual fingers. Switch prosthetics rely on the presence of a residual digits which can turn on and off small switches allowing for functionality.

On average, an upper extremity prosthetic can cost between $4000 and $8000 USDs, while the upper line externally powered prostheses can cost up to $50,000 USDs. Recently, 3D printed prosthetics have been developed. These can dramatically decrease the cost of the prosthetic while still allowing for increased functionality, such as movement of the thumb. Additionally, these 3D prosthetics may utilize open source software and/or hardware, allowing anyone to download and print them. Currently, a fully functional 3D printed prosthetic hand costs approximately $300 USDs to print.

There is no agreed upon standard of care for upper limb amputations. It is up to both the patient's discretion as well as their insurance company to decide whether they will be treated with a prosthetic. Even after a patient is given a prosthetic, they may choose not to use it. Almost half of the instances where a prosthetic is voluntarily not utilized, is simply due to the discomfort of the socket.

Sockets are necessary components of many prosthetics and allow for attachment of the myoelectric sensors as well as provide a comfortable interface between the patient and the prosthetic. In transradial amputees, the socket alone acts a support for the prosthetic as well. The current standard of designing the prosthetic is to make a plaster cast of the residual limb. This is almost always done by a certified prosthetist. Casting and measuring the residual limb not only allows for development of a comfortable socket but may also be necessary to determine appropriate size of the prosthetic as well. Additional methods of obtaining a model of the residual limb can be utilized. Currently a CAD (computer-aided design) hand scanner can be used to map out the residual limb. The CAD photometric method has been shown to contribute the least variance in inter and intra observer reliability when determining volume. Simple circumferential measurements using a measuring tape with a tensioning device was shown to be the least reliable. A CAD hand scanner can cost over $10,000 USDs.

Described herein is a scanning system that can determine the diameter of a residual limb. The scanning system comprises a scanner apparatus is in communication with a computing device comprising at least a processor and a memory. As used herein, processor refers to a physical hardware device that executes encoded instructions for performing functions on inputs and creating outputs. The processor of the computing device executes computer-readable instructions that interpret the data received from the scanning apparatus and converts it to a usable measurement applicable to designing the prosthetic. For example, the usable measurement may be the diameter of the residual limb. The computing device may also execute computer-readable instructions into a prosthetic design, which can be sent to a 3D printer for creation of the prosthetic, or at least a portion of it. Generally, the scanner apparatus comprises multiple sensors at known intervals, each connected to a rigid arm that can substantially encircle a residual limb to obtain measurements of the residual limb. Optionally or alternatively, various types of sensors may be used such as laser measuring devices and time-of-flight (TOF) sensors.

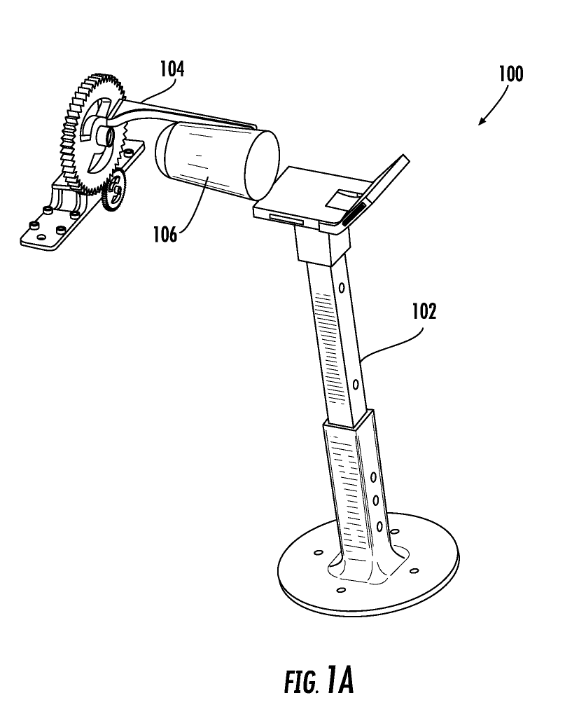

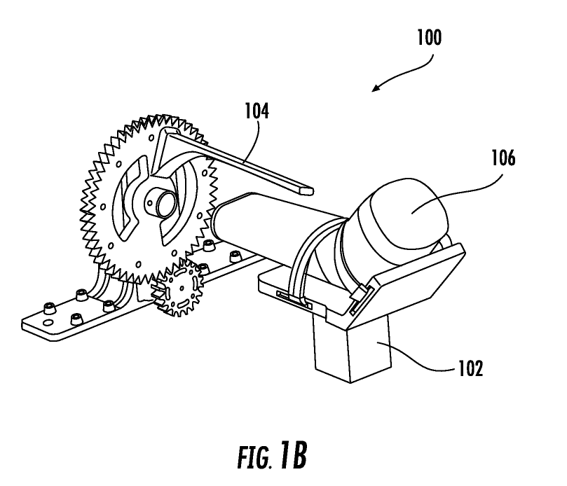

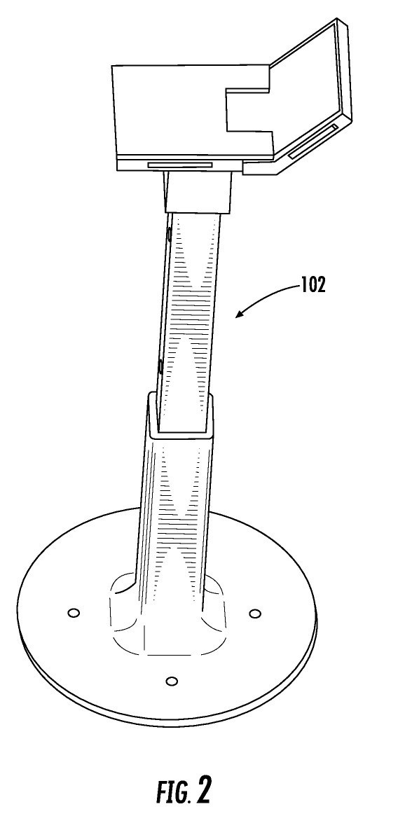

FIGS. 1A and 1B illustrate an exemplary overview system for scanning a residual limb and socket/prosthetic hyper-customization methods. The scanning system 100 comprises a stand 102 for the patient to place their residual limb as well as a scanner apparatus 104. The stand 102 functions to both center a patient's limb 106 in the scanner 104 as well as to stabilize it during scanning. The stand 102 additionally provides comfortable positioning to the user during the scanning process. An example of a design for a stand 102 is shown as FIG. 2, though other designs are contemplated within the scope of this disclosure.

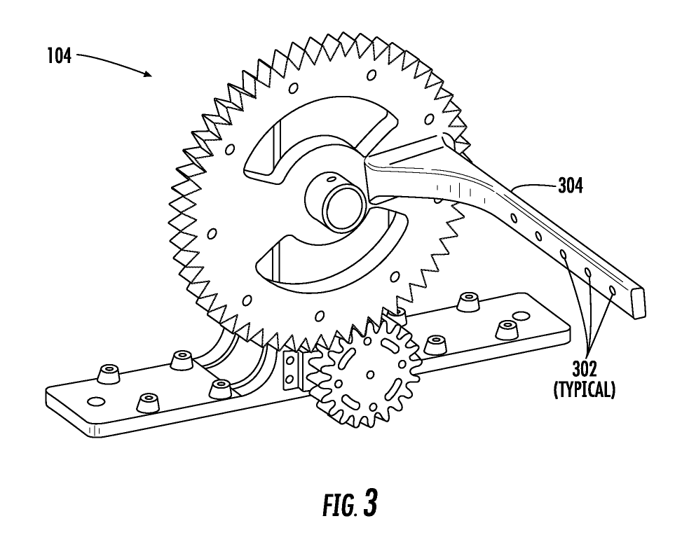

Referring to FIGS. 1A, 1B and 3, embodiments of the scanner apparatus 104 comprise one or more distance sensors 302, such as TOF sensors, mounted at fixed increments along a rigid arm 304, which is oriented parallel in relation to the residual limb. A servo motor (not shown in FIG. 3) turns the scanner arm 304 on command, which allows the one or more distance sensors 302 on the rigid arm 304 to rotate around the residual limb and record distance to surface of the residual limb as a function of scanner arm angle. For example, positioning the scanner arm 304 at top dead center (TDC) may be considered as 0° and 360°, and distance measurements can be made at discrete points (for example, every)5° by the one or more distance sensors 302 as the scanner arm 304 encircles the limb. Alternatively or optionally, distance measurements may be made continuously by the one or more distance sensors 302 as the scanner arm 304 encircles the limb. Once the distance measurements are obtained, a constant distance offset value can be subtracted from the measured distances to account for growing room and assure that the socket will be comfortable. Alternatively, a constant distance offset value can be added to the measured diameter of the residual limb to account for growth of the residual limb. Therefore, the usable measurement of the limb, as determined only by measurement, can be adjusted by a constant distance offset value to allow for growth of the residual limb. A second or higher order function can smoothly connect distance measurements around the limb giving accurate cross sections of the user's forearm along fixed increments. These cross sections can be connected along common angular increments and uniformly offset based on desired socket thickness to create a closed form 3D geometry that can comfortably fit to a patient's residual limb. Once this primary socket geometry is established, standardized 3-D features are automatically integrated into the existing model for the purposes of both harnessing a socket to patient and fastening the socket to a prosthetic limb.

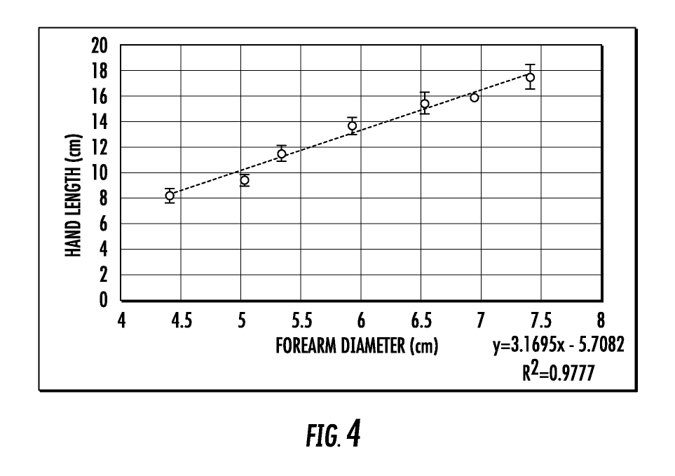

One anatomical constraint used to size the prosthetic limb to treat transradial amputations is the cross sectional diameter around the forearm. Anatomical constraints are defined as specific anthropometric measurements which are used to design a prosthetic. Such constraints can be utilized for appropriately sizing the prosthetic as well as designing it. In this case, determining the appropriate size of the prosthetic's components relies on a correlation made between the diameter of the forearm and the normal size of a human hand as a function of age. One such correlation is graphically illustrated in FIG. 4. The correlation assures proportionality between the prosthetic hand and the patient's other anthropometric measurements. The measurements used to obtain this correlation were documented by the U.S. Food and Drug Administration (FDA). Regression analysis shows the correlation to be y (hand length)=3.1695x−5.7082, where x is the measured forearm diameter and R²=0.9777. Table 1, below, provides anthropometric measurements for transradial amputations. These measurements, and/or the correlation, can be coded into the computer-executable instructions to provide a design for prosthetics having proportional measurements based on a measurement of forearm diameter as determined by the scanner apparatus. Once designed, the computing device can create at least a portion of the prosthetic using a 3D printer. Similarly, such measurements can be developed for legs for the design and creation of leg and/or foot prosthetics.

TABLE 1

| | Forearm | Hand |
|---|---|---|
| Age | Diameter | Length |
| (Months) | (cm) | (cm) |
| 3 | 4.203822 | 7.8 |
| 6 | 4.617834 | 8.7 |
| 9 | 5 | 9.4 |
| 12 | 5.063694 | 9.5 |
| 18 | 5.22293 | 10.1 |
| 24 | 5.350318 | 10.8 |
| 30 | 5.22293 | 11.3 |
| 36 | 5.350318 | 11.7 |
| 42 | 5.414013 | 12 |
| 48 | 5.477707 | 12.3 |
| 54 | 5.541401 | 12.7 |
| 60 | 5.764331 | 13 |
| 66 | 5.764331 | 13.4 |
| 72 | 5.987261 | 13.6 |
| 78 | 6.019108 | 14.1 |
| 84 | 6.082803 | 14.5 |
| 96 | 6.401274 | 15 |
| 108 | 6.656051 | 15.9 |
| 120 | 6.942675 | 15.9 |
| 132 | 7.261146 | 17 |
| 144 | 7.420382 | 17.2 |
| 156 | 7.643312 | 18.3 |

### Anthropometric Measurements for Transradial Amputations

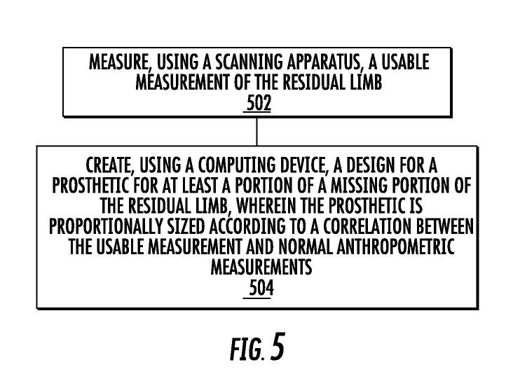

FIG. 5 is a flowchart that illustrates an exemplary method for developing a custom design for a prosthetic for a residual limb. The method comprises 502, measuring, using a scanning apparatus, a usable measurement of the residual limb. The scanning apparatus may comprise a rigid arm, wherein one or more measurement sensors are mounted on the rigid arm and the rigid arm with the one or more measurement sensors mounted thereon encircles the residual limb to measure the usable measurement. The one or more measurement sensors may comprise, for example, one or more time of flight (TOF) sensors. The rigid arm may be driven by, for example, an electric servo-motor or any other drive. Alternatively or optionally, the rigid arm may be cause to encircle the residual limb by hand using for example, a hand-crank. The one or more measurement sensors are in communication with a computing device. Such communication may comprise wired (including fiber optic) and/or wireless connections. For example, the sensors may use wireless Bluetooth communications, wireless fidelity (WiFi, as represented by any existing or future developed standards under IEEE 802.11n, where “n” is a version number of the standard, with such standards incorporated by reference) to transfer data to and/or receive data and/or instructions from the computing device. In one example, the computing device may be mounted on the rigid arm.

The computing device receives the measurement data from the one or more measurement sensors and 504, creates, using the computing device, a design for a prosthetic for at least a portion of a missing portion of the residual limb, wherein the prosthetic is proportionally sized according to a correlation between the usable measurement and normal anthropometric measurements. In one example, the usable measurement may comprise a diameter of the residual limb. For example, the usable measurement may comprise the diameter of a forearm and the prosthetic comprises at least a hand that is proportionally sized according to a correlation between the forearm diameter and hand size. Alternatively or optionally, the residual limb may be placed in a stand before measuring, using the scanning apparatus, the usable measurement of the residual limb. Also alternatively or optionally, the method may further comprise the computing device executing computer-readable instructions to create the designed prosthetic using a 3-dimensional (3D) printer.

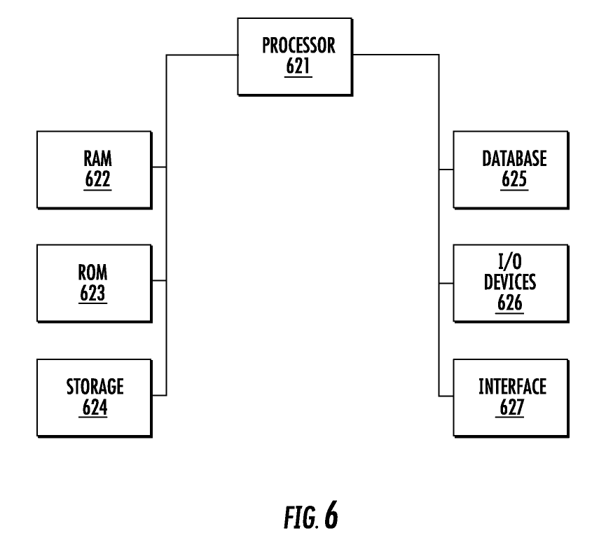

The system has been described above as comprised of units. One skilled in the art will appreciate that this is a functional description and that the respective functions can be performed by software, hardware, or a combination of software and hardware. A unit can be software, hardware, or a combination of software and hardware. The units can comprise software for developing a custom design for a prosthetic for a residual limb. In one exemplary aspect, the units can comprise a computing device that comprises a processor 621 as illustrated in FIG. 6 and described below.

FIG. 6 illustrates an exemplary computer that can be used for developing a custom design for a prosthetic for a residual limb. As used herein, “computer” may include a plurality of computers. The computers may include one or more hardware components such as, for example, a processor 621, a random access memory (RAM) module 622, a read-only memory (ROM) module 623, a storage 624, a database 625, one or more input/output (I/O) devices 626, and an interface 627. Alternatively and/or additionally, the computer may include one or more software components such as, for example, a computer-readable medium including computer executable instructions for performing a method associated with the exemplary embodiments. It is contemplated that one or more of the hardware components listed above may be implemented using software. For example, storage 624 may include a software partition associated with one or more other hardware components. It is understood that the components listed above are exemplary only and not intended to be limiting.

Processor 621 may include one or more processors, each configured to execute instructions and process data to perform one or more functions associated with a computer for developing a custom design for a prosthetic for a residual limb. Processor 621 may be communicatively coupled to RAM 622, ROM 623, storage 624, database 625, I/O devices 626, and interface 627. Processor 621 may be configured to execute sequences of computer program instructions to perform various processes. The computer program instructions may be loaded into RAM 622 for execution by processor 621.

RAM 622 and ROM 623 may each include one or more devices for storing information associated with operation of processor 621. For example, ROM 623 may include a memory device configured to access and store information associated with the computer, including information for identifying, initializing, and monitoring the operation of one or more components and subsystems. RAM 622 may include a memory device for storing data associated with one or more operations of processor 621. For example, ROM 623 may load instructions into RAM 622 for execution by processor 621.

Storage 624 may include any type of mass storage device configured to store information that processor 621 may need to perform processes consistent with the disclosed embodiments. For example, storage 624 may include one or more magnetic and/or optical disk devices, such as hard drives, CD-ROMs, DVD-ROMs, or any other type of mass media device.

Database 625 may include one or more software and/or hardware components that cooperate to store, organize, sort, filter, and/or arrange data used by the computer and/or processor 621. For example, database 625 may store correlation information for the usable measurement and the normal size for a human for the missing portion of the residual limb as a function of age. The database may also contain data and instructions associated with computer-executable instructions for developing a custom design for a prosthetic for a residual limb by causing a scanning apparatus to measure a usable measurement of the residual limb; receives measurement data from one or more measurement sensors of the scanning apparatus and create a design for a prosthetic for at least a portion of a missing portion of the residual limb, wherein the prosthetic is proportionally sized according to a correlation between the usable measurement and normal anthropometric measurements. Also alternatively or optionally, instructions may be stored that cause the computing device executing computer-readable instructions to create the designed prosthetic using a 3-dimensional (3D) printer. It is contemplated that database 625 may store additional and/or different information than that listed above.

I/O devices 626 may include one or more components configured to communicate information with a user associated with computer. For example, I/O devices may include a console with an integrated keyboard and mouse to allow a user to maintain a database of digital images, results of the analysis of the digital images, metrics, and the like. I/O devices 626 may also include a display including a graphical user interface (GUI) for outputting information on a monitor. I/O devices 626 may also include peripheral devices such as, for example, a 3D printer for printing at least a portion of the designed prosthetic, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 627 may include one or more components configured to transmit and receive data via a communication network, such as the Internet, a local area network, a workstation peer-to-peer network, a direct link network, a wireless network, or any other suitable communication platform. For example, interface 627 may include one or more modulators, demodulators, multiplexers, demultiplexers, network communication devices, wireless devices, antennas, modems, and any other type of device configured to enable data communication via a communication network.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method for developing a custom design for a prosthetic for a residual limb comprising:
measuring, using a scanning apparatus, a usable measurement of a residual limb; and
creating, using a computing device, a design for a prosthetic for at least a portion of a missing portion of the residual limb, wherein the prosthetic is proportionally sized by the computing device according to a correlation between the usable measurement and normal anthropometric measurements.

2. The method of claim 1, wherein the scanning apparatus comprises a rigid arm, wherein one or more measurement sensors are mounted on the rigid arm and said rigid arm with said one or more measurement sensors mounted thereon encircles the residual limb to measure the usable measurement.

3. The method of claim 2, wherein the one or more measurement sensors comprise one or more time of flight (TOF) sensors.

4. The method of claim 1, wherein the scanning apparatus is communicatively coupled with the computing device such that said usable measurement is transmitted from the scanning apparatus to the computing device.

5. The method of claim 1, wherein the usable measurement comprises a diameter of the residual limb.

6. The method of claim 5, wherein the usable measurement comprises the diameter of a forearm and the prosthetic comprises at least a hand that is proportionally sized according to a correlation between the forearm diameter and hand size.

7. The method of claim 1, wherein the residual limb is placed in a stand before measuring, using the scanning apparatus, the usable measurement of the residual limb.

8. The method of claim 1, further comprising the computing device executing computer-readable instructions to create the designed prosthetic using a 3-dimensional (3D) printer.

9. The method of claim 1, further comprising adjusting the usable measurement of the residual limb by a constant distance offset value to allow for growth of the residual limb.

10. A system for developing a custom design for a prosthetic for a residual limb comprising:
a scanning apparatus, wherein the scanning apparatus determines a usable measurement of the residual limb; and
a computing device comprising a processor and a memory, the processor executing computer-readable instructions stored in the memory, said computer-readable instructions causing the processor to:
receive the usable measurement from the scanning apparatus; and
create a design for a prosthetic for at least a portion of a missing portion of the residual limb, wherein the prosthetic is proportionally sized according to a correlation between the usable measurement and normal anthropometric measurements.

11. The system of claim 10, wherein the scanning apparatus comprises a rigid arm, wherein one or more measurement sensors are mounted on the rigid arm and said rigid arm with said one or more measurement sensors mounted thereon encircles the residual limb to measure the usable measurement.

12. The system of claim 11, wherein the one or more measurement sensors comprise one or more time of flight (TOF) sensors.

13. The system of claim 10, wherein the scanning apparatus is communicatively coupled with the computing device such that said usable measurement is transmitted from the scanning apparatus to the computing device.

14. The system of claim 10, wherein the usable measurement comprises a diameter of the residual limb.

15. The system of claim 14, wherein the usable measurement comprises the diameter of a forearm and the processor executes computer-readable instructions to design a prosthetic that comprises at least a hand that is proportionally sized according to a correlation between the forearm diameter and hand size.

16. The system of claim 10, further comprising a stand, wherein the residual limb is placed in a stand before measuring, using the scanning apparatus, the usable measurement of the residual limb.

17. The system of claim 10, further comprising a three-dimensional (3D) printer, wherein the processor executes computer-readable instructions to create the designed prosthetic using the 3D printer.

18. The system of claim 10, further comprising the processor executing computer-readable instructions to adjust the usable measurement of the residual limb by a constant distance offset value to allow for growth of the residual limb.

19. A non-transitory computer-readable medium having computer executable instructions stored thereon, said computer executable instructions causing a processor to perform a method of developing a custom design for a prosthetic for a residual limb, said method comprising:
receiving a usable measurement of a residual limb from a scanning apparatus; and
creating a design for a prosthetic for at least a portion of a missing portion of the residual limb, wherein the prosthetic is proportionally sized according to a correlation between the usable measurement and normal anthropometric measurements.

20. The non-transitory computer-readable medium of claim 19, further comprising computer-readable instructions to create the designed prosthetic using a 3-dimensional (3D) printer.
